(19) United States
(12) Patent Application Publication
(10) Pub. No.: US 2012/0310177 A1
(43) Pub. Date: Dec. 6, 2012

(54) CONNECTOR SYSTEM FOR NUTRIENT CONTAINERS FOR ENTERAL NUTRITION

(75) Inventors: Michael Becker, Knittlingen (DE); Barbara Breuer-Thal, Idstein (DE)

(73) Assignee: FRESENIUS KABI DEUTSCHLAND GMBH, Bad Homburg (DE)

(21) Appl. No.: 13/512,611

(22) PCT Filed: Dec. 10, 2010
(86) PCT No.: PCT/EP2010/069359

§ 371 Date: Aug. 3, 2012

Related U.S. Application Data
(60) Provisional application No. 61/321,880, filed on Apr. 8, 2010.

(30) Foreign Application Priority Data
Dec. 10, 2009 (EP) 09015292.7

(51) Int. Cl. A61M 31/00

(52) U.S. Cl. 604/244; 604/257

(57) ABSTRACT

The present invention discloses an attachment (1) for a device for enteral feeding, which comprises at least two connecting pieces (2, 5), wherein the first connecting piece (2) can be connected to the connection nozzle (3) of a feeding container (4), and the second connecting piece (5) can be connected to a transfer unit (6) so that a food flow from the feeding container through the connection nozzle and the attachment into transfer unit is made possible. Furthermore, the attachment comprises a means for opening (7) of the feeding container. Furthermore, a device for enteral feeding comprising the attachment according to the invention is disclosed.

### TECHNICAL FIELD

The present invention relates to the field of enteral feeding. In particular, a special attachment for a device for enteral feeding is disclosed. The invention further relates to a device for enteral feeding with such attachment.

### PRIOR ART

In enteral feeding, the patient is administered a liquid nutrient solution with an enteral feeding tube. The enteral nutrient solution is provided in a feeding container, which is connected by a so-called transfer unit to an enteral feeding tube. The transfer unit has a probe tube with distal and proximal connecting pieces. The distal connecting piece is connected to the feeding tube and the proximal connecting piece is connected to the feeding container so that the nutrient solution flows from the feeding container through the connecting tube of the transfer unit into the feeding tube.

The feeding container is usually replaced several times a day, while for the transfer unit, a change every 24 hours is recommended. The feeding tube can be used up to several months.

To connect the transfer unit with the feeding tube, in the prior art, the luer lock connectors are used so that the transfer unit and the feeding tube can be connected to each other without the risk that the probe tube of the feeding tube inadvertently detaches itself from the connecting tube of the transfer unit. Alternatively, also so-called adapter skirts are used, which are connected distally to the transfer unit, and which are connected with semi-rigid funnel attachments to the proximal end of the feeding tubes by means of a press-fit connection. An improved connection between the transfer unit and the probe tube has been described in WO 2008/049568 A.

The type of connection of the feeding container to the transfer the unit depends, among other things, on the feeding container used, because various manufacturers of such feeding containers offer different technical solutions. In many cases, a so-called spike, i.e. a “mandrel” or cannula is used, which is connected to the dispensing part (port) of the feeding container. In this case, the port is formed as a connection nozzle and serves both as a channel for the spike and a fastening means for the transfer unit. Here, the spike penetrates the sealing of the feeding container, or it penetrates a membrane that closes again when the spike is removed. For example, with the feeding containers manufactured by Nestle, the connection nozzle is applied directly to the completely sealed feeding container feeding, whereas in the case of the manufactures Danone and Fresenius Kabi, it is welded into the film of the sealed feeding container. In the case of the latter two manufacturers, therefore, a sealing membrane or a sealing cap is applied to the end of the connection nozzle that faces the feeding container in order to keep the food contained in the container free of germs.

A disadvantage, however, is that this connectivity between the feeding container and the transfer unit requires a spike to pierce either the wall of the feeding container or a sealing attached to the connection nozzle. In this sense, it poses a significant risk to the patient, because other connection systems that are used in particular in the intravenous application also have a spike and therefore in principle can be used to connect to the feeding container, if they are then connected by adapters to the enteral feeding tubes. Should, for example, an enteral feeding solution be erroneously administered intravenously established access, this would pose a real danger to the life of the patient.

### SUMMARY OF THE INVENTION

The technical task underlying the invention is the provision of an universally usable, secure and, at the same time, easy-to-use connection system for feeding containers for enteral feeding, as well as a device for enteral feeding with such connection system.

The solution of these tasks is provided by the features indicated in the patent claims 1, 8 and 10. Advantageous embodiments are specified in the dependent claims, the description and the drawings.

The inventive attachment for a device for enteral feeding comprises at least two connecting pieces, wherein the first connecting piece is connectable to the connection nozzle of the feeding container, and the second connecting piece is connectable to a transfer unit so that a food flow is made possible from the feeding container through the connection nozzle and attachment to the transfer unit, where the attachment comprises a means for opening the feeding container.

The core idea of the present invention is

that the proximal connector of the transfer unit has no longer any spike, or is a connector having a spike incompatible with a feeding container, which is equipped with an attachment,

that the proximal connector of the transfer unit can be connected to the inventive attachment and forms then a unit closed to the environment, which protects the food from contamination,

that the proximal connector of the transfer unit can be connected to the inventive attachment of the feeding container, before the seal of the feeding container is opened, in order to minimize the risk of leakage and the risk of contamination,

that in the inventive attachment, there is integrated at least one, preferably mechanical, means for opening the seal of the feeding containers,

that the inventive attachment is designed such that a spike designed for intravenous applications is not suitable to open the feeding container and its seal by the attachment.

This is inventively achieved in that the attachment serves as a link between the transfer unit and the feeding container, and, on the one hand, can be connected both to the nozzle of the feeding container and the transfer unit, and, on the other hand, a means for opening the feeding container is enclosed by the attachment. Thus, the present invention advantageously allows to connect a feeding container to a transfer unit without having to use a spike connected to the tube of the transfer unit. As a result, it is excluded by the present invention that other types of connection used in the clinical environment—for example, for intravenous administration—that comprise a spike could be connected to the feeding container. Thus, a substantial increase in safety for the patient is achieved.

The device for enteral feeding can be any device known in the art and suitable for such a purpose. In an embodiment the device comprises, in addition to the inventive attachment, at least one feeding container, to which preferably a connection nozzle is mounted, and a transfer unit and a feeding tube.

The feeding container can be any container suitable for enteral feeding. In the prior art, there are a number of such feeding containers, which are typically designed in a bag form or as a semi-rigid container and are preferably made of plastic.

A person skilled in the art knows likewise various suitable embodiments of the connection nozzle, all of which should be considered to be included in the present invention. The connection nozzle is called “port” and serves in a preferred embodiment as a dispensing and/or filling part. The connection nozzle can therefore be suitable for filling the feeding container with food and/or for dispensing the food from the container. Another function of the connection nozzles is to provide a channel for the spike. In a preferred embodiment, the connection nozzle provides an attachment means for the inventive nozzle attachment and/or a closure or cap attachment.

In another embodiment, the connection nozzle consists of a connection component, which is glued or welded with the bag film, and a hollow cylindrical, preferably rigid portion, wherein the hollow cylindrical part can be brought into connection with the attachment.

The connection between the connection nozzle and the feeding container can be accomplished in any manner known to a person skilled in the art. In a preferred embodiment, the connection nozzle is mounted directly to the wall of the feeding container, e.g., by gluing. In this way, there is no direct access to the food contained in the container, i.e., the feeding container wall itself acts as a sealer. In a further preferred embodiment, the connection nozzle is welded into the bag seam of feeding container. This has the advantage that a more secure attachment of the connection nozzle to the feeding container is achieved and the bag can be filled through the connection nozzle.

In a further embodiment, the connection nozzle comprises at least one sealing means. Such a sealing means has the purpose and advantage that contamination is avoided in the container that contains food. Although all suitable sealing means known in the art are regarded as being included in the invention, in a preferred embodiment the sealing means is a membrane, film or aluminum foil, which is applied to the end of the connection nozzle that is on the side opposite to the feeding container. Even more preferably, the sealing means is a clip-on or screw-on closure cap, which to the end of the connection nozzle that is on the side opposite to the feeding container. This cap contains a membrane that, on the one hand, serves as a sealing element between the closure cap and the connection nozzle, and, on the hand, fulfills the function of a valve. Such a membrane allows to reversibly open access to the food in the container and close it again. This is made possible, for example, by a membrane with star-shaped, central incisions, which after the withdrawal of the spike close the opening to the food. Such a membrane offers the advantage that a food bag can be connected by the transfer unit and removed again, especially when the feeding container is hanging on an infusion stand. Those skilled in the art know a variety of such membranes with reversible valve function, for example, the membrane contains in the enteral feeding bags marketed by Fresenius Kabi under the name of EasyBag. In a further preferred embodiment, the connection nozzle comprises an inner membrane, preferably with a reversible valve function, and a sealing film overlying the membrane, preferably an aluminum foil.

The transfer unit can be any transfer unit suitable for the purpose of enteral feeding. Exemplary transfer units are commercially available from Fresenius Kabi under the trade name of “APPLIX”. The transfer unit provides the link between the food bag and the feeding tube, and comprises a probe tube with a distal and proximal connecting piece. Using the distal connecting piece, it can be connected to the feeding tube, and over the proximal connecting piece, it can be connected by the invented attachment to the feeding container. The transfer unit preferably comprises a pump to transport the food from the feeding container through the feeding tube into the patient. The transfer unit is preferably a DEHP-free system. The feeding tube can be any feeding tube known to a person skilled in the art, preferably a nasogastric tube or gastrostomy tube.

The attachment of the present invention forms the connection between the connection nozzle and the transfer unit. This connection is achieved about by the at least two connecting pieces of the attachment. The connecting pieces are formed such that a food flow is allowed between the connection nozzle and the transfer unit. To this end, the connecting pieces preferably have at least one opening, wherein the openings of the connecting pieces are in contact with each other, for example, by a cavity formed as a channel, and allow the flow of food from one opening to another.

In a preferred embodiment of the invention, the attachment according to the invention comprises exactly two connecting pieces. In another preferred embodiment, the attachment comprises 3, 4, 5, or more than 5 connecting pieces, wherein at least one connecting piece can be connected to the transfer system and at least one connecting piece can be connected to the connection nozzle. Even more preferably, with more than 2 connecting pieces, all except one can be connected to the connection nozzle of a feeding container. This has, inter alia, the advantage that the patient can be simultaneously supplied by a single feeding tube with possibly different food from more than one feeding container.

The attachment can be made of any suitable material, when plastics are preferred.

In a further preferred embodiment, at least one connecting piece of the attachment is formed as a seal, which preferably creates a secure and positive connection between the attachment and the transfer system and/or the attachment and the connection nozzle. A seal advantageously prevents leakage of food and contamination of food through the place of connection between the attachment and the connection nozzle and/or the attachment and the transfer unit.

The first connecting piece, which can be connected to the nozzle, is preferably designed such that it allows an irreversible connection between the attachment and the connection nozzle. More preferably, the manufacturer supplies the feeding containers already connected to the attachment according to the invention. This has the advantage that with such a feeding container with pre-mounted attachment, the user can no longer connect a transfer unit comprising a spike as a connecting element via the connection nozzle to the feeding container. For example, this prevents the connection of a transfer unit that is used for intravenous application.

A person skilled in the art knows a variety of ways to ensure an irreversible connection between the attachment and the connection nozzle. The term “irreversible connection” is here preferably understood in that the user cannot detach the connection without the use of excessive force and/or the use of tools. In preferred embodiments, either the attachment or the connection nozzle comprises a snap-in edge, which forms an irreversible connection with a corresponding projection of the connection nozzle or attachment that is to be connected. This has the advantage that the attachment can be simply slipped over the nozzle—or vice versa—and an irreversible connection is immediately established. Other alternatives or additional ways to establish an irreversible connection are gluing and/or welding.

In a particularly preferred embodiment of the invention, an irreversible connection between the connection nozzle of the feeding container and the attachment according to the invention is already performed prior to delivery of the feeding container to the consumer or user. This has the advantage that it is ensured that the consumer/user must always use a connector complementary to the second connecting piece of the attachment in order to open the feeding container. Thus, it is ensured that connection can be detached only without using a spike.

In another preferred embodiment, an irreversible connection between the attachment and the connection nozzle is achieved by means of a glue, which can also be used in addition.

The second connecting piece that is connectable to the transfer unit is preferably configured such that a connection that is simple to establish and detach is established with the transfer unit. Even more preferably, this second connecting piece comprises a locking element, which is formed such that a positive and releasable connection is established with a connecting piece of the transfer unit, which comprises a locking element that is complementary to the locking element of the second connecting piece. The locking element can be any suitable embodiment known to a person skilled in the art. As for locking elements, basically all positive detachable connections are possible, such as connectors, screwed connections, especially the sleeve caps, screw caps, bayonet joints, or the like.

In a particularly preferred embodiment, the locking element of the second connecting piece is formed as a screw cap with an internal thread.

In another particularly preferred embodiment, the second connecting piece of the attachment according to the invention is designed conically or step-shaped with several sections of different diameters and comprises a locking element that is configured such that, on the one side, a releasable positive connection to a connecting piece of a transfer unit can be established, which comprises a locking element that is complementary with the locking element of the second connecting piece, and that, on the other side, a detachable connection is possible with funnel-shaped connecting pieces of different diameters of different transfer units.

Even more preferably, the second connecting piece of the attachment according to the invention is designed as a male or female “ENLock” connector. This allows for quick and secure connection with the transfer unit, if its connecting piece is designed as a complementary female or male “EN Lock” connector. This makes possible a quick and easy release of such a connection. The “ENLock” connection system is well known to a person skilled in the art, and is described, among others, in WO 2008/049568 A, which is herein incorporated in its entirety into the present application, and is available for visual inspection as a draft of the ISO working group—TC210/JWG4, PG 3—Enteral Connectors.

The formation of the second connecting piece of the attachment according to the invention as an “ENLock” connector offers, in addition to an easy-to-use and secure connection, among other things, the advantage of significant cost savings with the transfer unit, and that the handling of the transfer unit is facilitated, because here, in addition to the already existing distal “ENLock” connector, there is used just another “ENLock” connector as a proximal connecting element and, therefore, the frequently exchanged transfer units no longer need to be equipped with means for opening the feeding container.

In another preferred embodiment, the attachment further comprises at least one means for sealing at least one of the connecting pieces, preferably all connecting pieces. This has the advantage that the interior of the attachment can be kept sterile. Even more preferably, the attachment comprises means for the sealing of the second connecting piece. This has the advantage that after putting on the attachment and prior to connection to the transfer unit, the feeding container is closed and a contamination of the food is prevented. A person skilled din the art knows a plurality of possible sealing means that can be used for the sealing of the second connecting piece. Preferred means include connectors, threaded connections, sleeve caps, screw caps, bayonet connectors, membranes, films, in particular aluminum foils, slip-on, screw-on and/or tear-off caps.

The attachment according to the present invention further comprises means for opening the feeding container. Preferably the means for opening is integrated in the attachment, even more preferably, after the attachment is connected to the nozzle, the means is no longer visible and/or accessible from outside.

The means for opening the feeding container can be any suitable means. In a preferred embodiment, it is a means for piercing the sealing of the feeding container; even more preferably, it is a spike, or one or more mandrels, or a cannula. As initially mentioned, there are available feeding containers from different vendors that vary in terms of the sealing and thus in the process of opening them. Accordingly, the means for opening the feeding container will also be designed differently. Based on the particular sealing of the respective container, a person skilled in the art will be able to easily adapt the means for opening it.

In an embodiment, the connection nozzle is mounted directly onto the food container wall, as is the case, for example, with the feeding containers of the manufacturer Nestle. In this case, the feeding container wall itself serves as a sealing of the food container. Accordingly, the means for opening the feeding containers must be suitable to pierce the feeding container wall. Furthermore, the means for opening must have a sufficient length to reach from the opening of the connection nozzle at least up to behind the feeding container wall. In a preferred embodiment, the means for opening the feeding container made in one piece with the attachment and has the form of a cannula which, even more preferably, is arranged centrally in the attachment.

In another embodiment, the connection nozzle is welded into the seam of the feeding container film, as is the case, for example, with the food containers of the manufacturers Danone or Fresenius Kabi. In these cases, the connection nozzle is sealed on its end pointing away from the feeding container by a sealant, preferably by a membrane and/or a film, such as aluminum foil. Accordingly, the means for opening the feeding container must be suitable to pierce the sealing(s). Furthermore, compared to above embodiment, the means for opening need only have a smaller length in order to reach from the opening of the connection nozzle at least up to behind the feeding container wall. In a preferred embodiment, the means for opening the feeding container is made in one piece with the attachment and has the form of a spike, or one or more mandrels or a cannula, which, even more preferably, is arranged centrally in the interior of the attachment.

In another embodiment, the means for opening the feeding container can be moved in the direction of the first connecting piece, i.e., in the direction of the sealing of the feeding container. Preferably, this movement is an axial movement. The means for opening the feeding container can take up at least two defined positions: a first one, in which it does not pierce the sealing of the food container, i.e., the feeding container is closed, and a second one, in which it penetrates the sealing of the feeding container, i.e., the feeding container is opened. In addition to these two positions, other positions are also possible, for example, positions between these two aforementioned points, or, for example, stepless positions produced by a threaded connection between the attachment and the connection nozzle.

The attachment according to the invention can further comprise a means for locking the means for opening the feeding container, which holds the means for opening in at least one of the at least two defined positions. The preferred such means for locking have already been described above,

In a particularly preferred embodiment, the attachment comprises a thread that enables the movement of the means for opening by a rotational movement of the attachment. In this case, the thread of the attachment engages preferably in a complementary thread, which is enclosed by the connection nozzle. This has the advantage that the food container is easy to open. In particular, a thread provides at the same time a means for locking the means for opening the feeding container. The thread comprised by the attachment can be an internal or an external thread, preference is given to an internal thread. The connecting thread of the nozzle can therefore be an external or an internal thread and the preferred one is an external thread.

Another aspect the invention relates to an attachment for a device for enteral feeding, which comprises at least two connecting pieces, wherein the first connecting piece can be connected to the connection nozzle of a food container, and the second connecting piece can be connected to a transfer unit so that a food flow from the feeding container through the nozzle and the attachment to the transfer unit is made possible, wherein the second connecting piece is designed as an “NLock” connectors. In a preferred embodiment, a food bag with an attachment according to the invention irreversibly connected to the connection nozzle is made already in the production process. This ensures that, in order to open the feeding container, the consumer/user must always use a connector that is complementary with the second connecting piece of the attachment. This ensures that only opening solutions without a spike are used.

The embodiment of the attachment of this aspect of the present invention accordingly has no means for opening the food container, but otherwise can realize all the features described above. Such an attachment is suitable, for example, for feeding containers that do not have any sealing to be pierced. This allows for the connection nozzle of the feeding container to only be closed by a removable fastener on the nozzle, such as a screw, a flip-off cap and/or an applied film. After removal of this seal by the user, there is already a direct access to the food through a nozzle welded into the bag seam. In such a case, although the attachment according to the invention can also be used with the means for opening the food container, it is not absolutely necessary. The embodiment of the invention of the attachment without means to open the feeding container but with a second connecting piece designed as an “ENLock” connector has, therefore, the advantage, on the one hand, of achieving a cost reduction in making the attachment, but still ensuring, on the other hand, that based on the “ENLock” connector, it is not possible for user to connect to the food container other types of connections that are using a spike in the clinical environment, for example, intravenous applications.

The present invention further relates to a device for enteral feeding comprising the above-described inventive attachment and a feeding container with a connection nozzle attached thereto, said attachment and connection nozzle being connected to each other by the first connecting piece. Preference is given to the attachment and the connection nozzle being irreversibly linked.

It is obvious to a person skilled in the art that that all of the above characteristics, in particular with respect to the food container, the attachment, the connecting pieces, the connection nozzle, the transfer unit and/or the feeding tube are also applicable for the device for enteral feeding described here. It is therefore waived to list these features again. However, a few preferred embodiments will be explicitly mentioned.

In a preferred embodiment, the connection nozzle of the device comprises at least one sealing means, which is arranged on the end of the connection nozzle that points away from the feeding container and thus protects the contents of the feeding container from contamination. In a preferred embodiment, the at least one sealing means is a membrane, a film, an aluminum foil, a slip-on cap, a screw-on cap and/or a flip-off cap. In a further preferred embodiment, the connection nozzle comprises an inner membrane, preferably with a reversible valve function, and a film located above the membrane, preferably an aluminum foil.

In a further preferred embodiment, the attachment and the connection nozzle of the device comprise each at least one thread, which engage with each other and therefore allow for the movement of the means for opening toward the first connecting piece. Preferably, the means for opening is moved axially in this case and/or the movement is made possible by a rotational movement of the attachment. In a preferred embodiment, the axial movement can also be generated with a bayonet connection and/or be locked in.

In another preferred embodiment, the device for enteral feeding further comprises a transfer unit, wherein the transfer unit and the attachment are positively connected to each other by the second connecting piece of the attachment. Even more preferably, both the second connecting piece of the attachment and the connected connecting piece of the transfer unit are designed as a complementary “ENLock” connector. For example, the second connecting piece of the attachment is a male “ENLock” connector, while the connecting piece of the transfer unit is a female “ENLock” connector, or vice versa.

Another aspect of the invention relates to the use of the previously described attachment according to the invention or the device comprising the attachment for enteral feeding of an individual, for example, of a human or an animal.

Another aspect the invention relates to a method for providing a device for enteral feeding, comprising the step of connecting a feeding container and a transfer unit by means of the inventive attachment.

In an embodiment, the transfer unit is connected to the attachment, after the feeding container has been opened by the means for opening contained in the attachment. Here, before the connection to the transfer unit, and in spite of the piercing of the sealing of the feeding container, i.e., despite the opening of the feeding container, protection of the food in the container against contamination can be ensured, if the second connecting piece of the inventive attachment is sealed, for example, by a flip-off cap, a screwed-on cap or an aluminum film.

In a further embodiment, the transfer unit is connected to the attachment before the food container is opened by the means for opening contained in the attachment, but preferably after the attachment has been connected to the feeding container. This has the advantage that an overall system closed to the outside world is established before the food flow begins.

The following describes various embodiments of the invention with reference to the drawings.

### BRIEF DESCRIPTION OF THE DRAWING FIGURES

Shown are:

FIG. 1: the schematic structure of two common types of feeding containers.

FIG. 2: A first embodiment of the attachment or the device according to the invention.

FIG. 3: A first embodiment of the attachment or the device according to the invention

FIG. 4: A first embodiment of the attachment or the device according to the invention.

FIG. 5: A first embodiment of the attachment or the device according to the invention

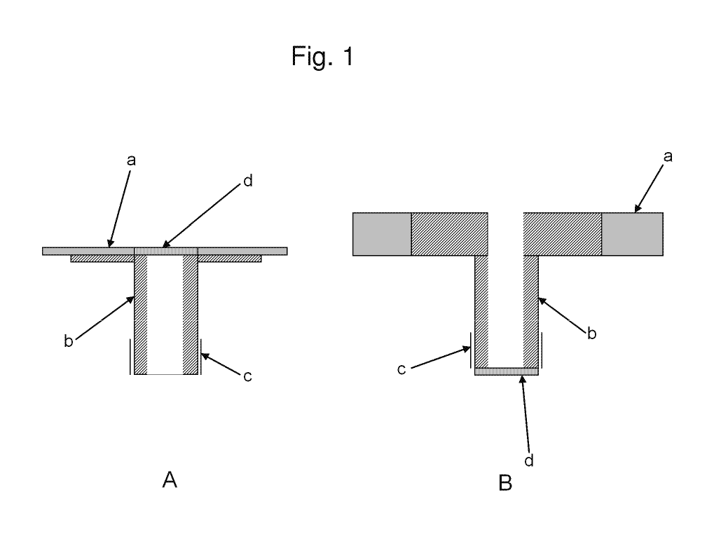

FIG. 1 shows the schematic structure of the two common variants of a container of food. Shown is a detail from the area of a food bag, in which the connection nozzle is brought into connection with the food bag.

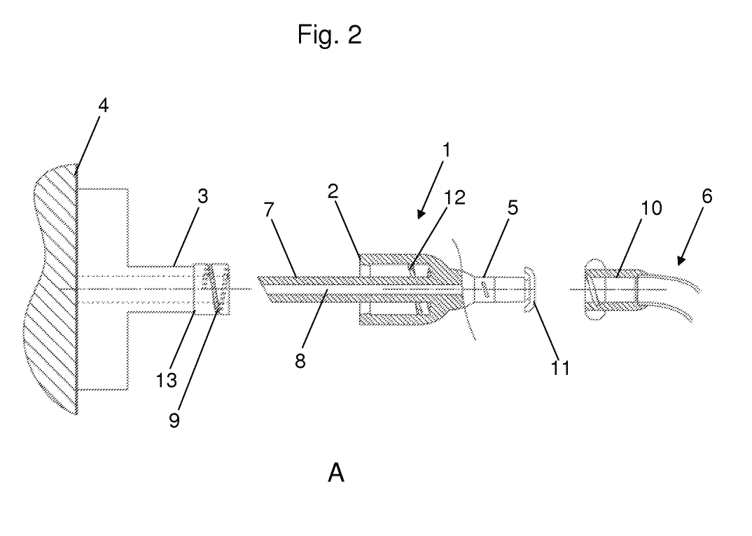

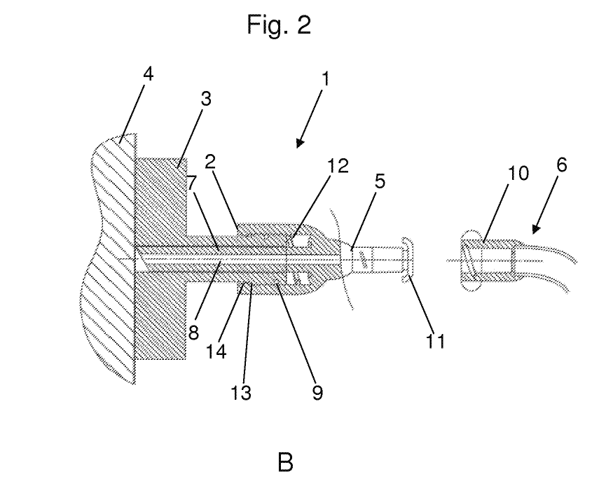

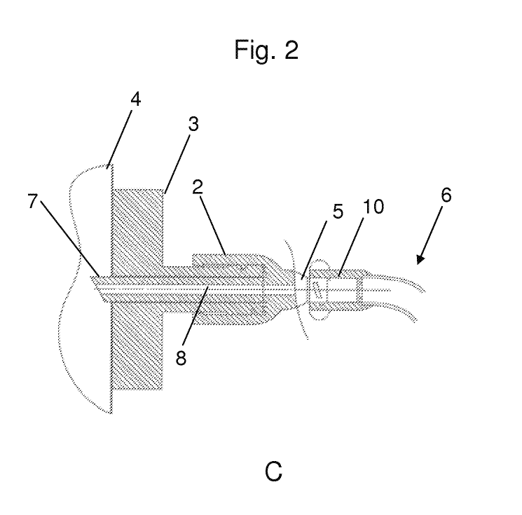

FIG. 1A shows a schematic of the food bag made by the manufacturer Nestle. There can be seen the food bag wall a, to which is glued the connection nozzle. The connection nozzle serves as a channel for the means for opening and as a dispensing part and has a thread c, by which the means for opening the attachment according to the invention can be moved. The irreversible connection of the invention attachment with the connection nozzle is preferably achieved by snap-in connection, which is shown in FIG. 2f.

Furthermore, it is seen the section d of the food bag that must be penetrated by the means for opening the attachment in order to open the food bag and to ensure the dispensing of the food. In the example shown here, therefore, no additional sealing is present, because the food bag wall itself is the sealing.

FIG. 1B shows a schematic of the food bag of the manufacturer Danone. One can see again the food prey wall a having a welded seam, into which the connection nozzle b is welded. The connection nozzle serves as a filling and dispensing part and has a thread c, by which the means for opening the seal integrated in the attachment according to the invention is axially displaceable. The irreversible connection of the attachment according to the invention to the connection nozzle is preferably achieved by means of a snap connection, which is shown in FIG. 2f.

One can further see the sealing d of the food bag, which must be pierced by the means for opening of the attachment in order to open the food bag and to ensure the dispensing of the food. In the example shown here, a sealing membrane was applied to the end of the connection nozzle pointing away from the food bag in order to avoid contamination of the bag contents.

FIG. 2 shows a first embodiment of the invention attachment or the invention device.

As shown in FIG. 2A, the attachment 1 for a device for enteral feeding comprises the two connecting pieces 2 and 5. The first connecting piece 2 can be connected to the nozzle 3 of a feeding container 4. The second connecting piece 5 can be connected to the transfer unit 6, said connecting piece 5 of the attachment being designed as the male “EN-Lock” connector, and the connecting piece 10 of the transfer unit, which is attached to the proximal end of the probe tube of the transfer unit, is formed as a female “ENLock” connector. The second connecting piece 5 of the attachment further comprises a sealing in the form of a flip-off cap 11.

As can be seen, the feeding container comprises no further sealing means but rather the connection nozzle is glued to the feeding container wall.

The attachment further comprises a means for opening the feeding container 7, which is formed as a long spike and comprises a cavity or channel 8 that connects the two connecting pieces 2 and 5 so that a flow of food from the feeding container through the connection nozzle and the attachment to the transfer unit is made possible. The attachment 1 and the means for opening are made in one piece of plastic.

The connection nozzle 3 also comprises an external thread 9, into which the internal thread 12 of the attachment engages. Thus, by a rotation of the attachment the means for opening the feeding container 7 can be moved axially in the direction of the first connecting piece and, after penetrating the food container wall, can be locked.

The connection nozzle 3 further comprises a projection 13, which corresponds to a snap-in edge of the attachment and makes possible an irreversible snap-in connection between the connection nozzle and the attachment, which is produced in the production process so that the channel of the connecting nozzle cannot be connected to a spike of an intravenous transfer unit. Furthermore, the connection between the connection nozzle and the attachment is sealed and the contents of the feeding container are effectively protected from contamination.

FIG. 2B shows the same embodiment as FIG. 2A, after the nozzle attachment 1 has been connected to the connection nozzle 3. The connection was achieved by simply plugging on the attachment on the connection nozzle in the production process, wherein the snap-in edge 14 of the attachment was pressed behind the projection 13 of the connection nozzle in order to ensure an irreversible connection.

Furthermore, one can see that the internal thread 12 of the attachment does not yet completely engage in the external thread 9 of the connection nozzle and the means for opening the feeding container 7 is only in a first defined position on the wall of the feeding container 4 without piercing it. In addition, the flip-off cap 11 is still attached to the second connection piece.

FIG. 2C shows the same embodiment as FIGS. 2A and 2B, after the flip-off cap 11 has been removed from the second connecting piece 5 and the male “ENLock” connector 5 of the attachment was connected to the female “ENLock” connector 10 of the transfer unit 6. This connection is characterized by an increased link security, while being easy to use. In particular, the connection of the transfer unit to the attachment can be made before the bag film is opened.

Furthermore, by a rotational movement of the attachment and by the mutual engagement of the two threads 9 and 12, the means for opening the feeding container 7 was axially moved in the direction of the feeding container 4 to a second defined position and it now penetrates the wall of the feeding container. This ensures a hermetically sealed food flow from the feeding container 4 through the means for opening the attachment 1, further through the “ENLock” connection 5, 10 to the transfer system and the risk of contamination is minimized.

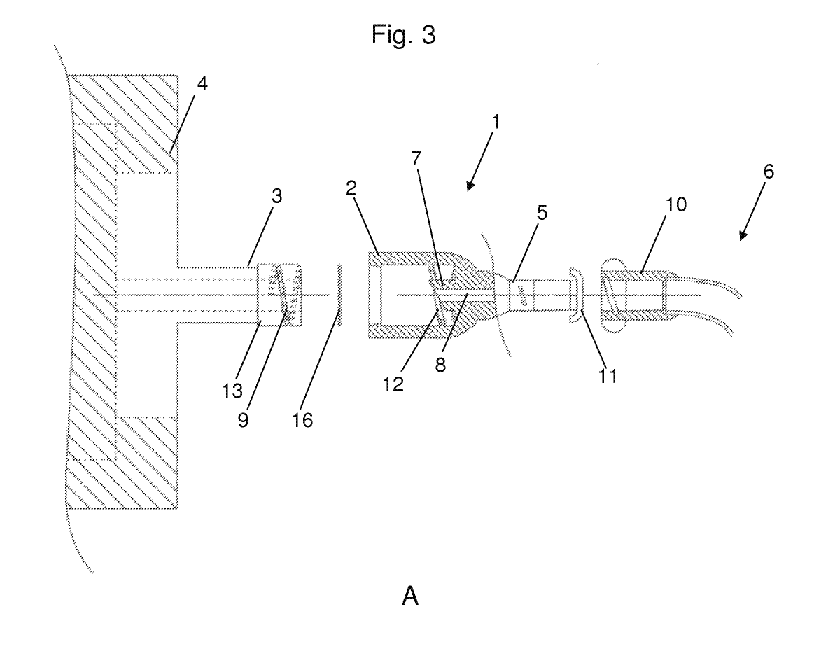

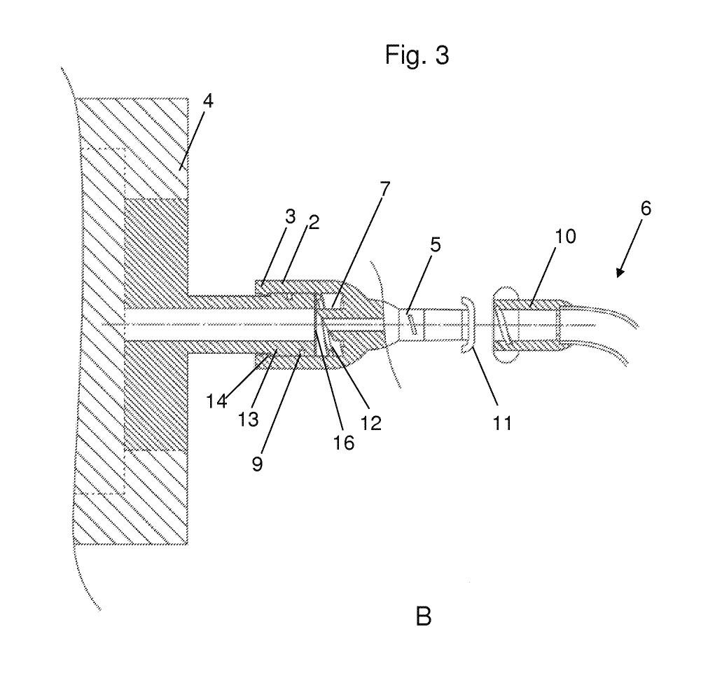

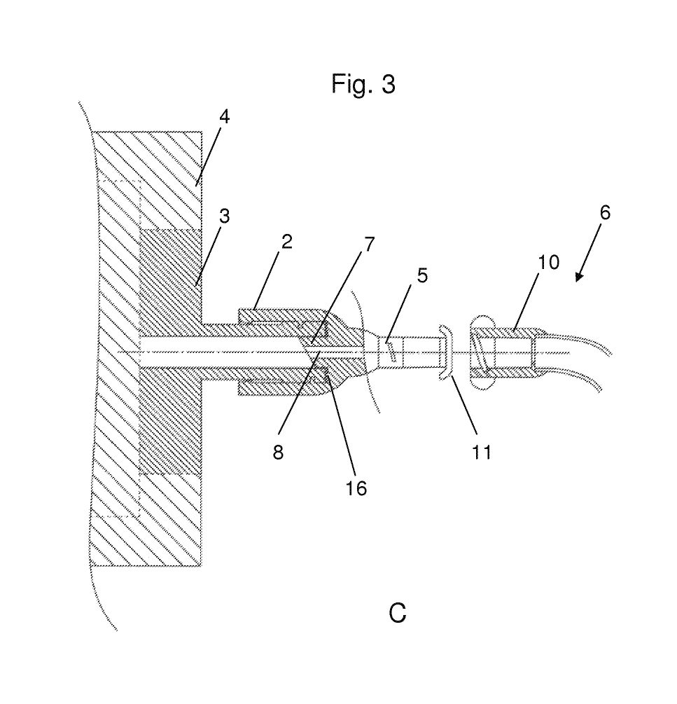

FIG. 3 shows a further embodiment of the attachment or the device according to the invention.

As shown in FIG. 3A, the attachment 1 for a device for enteral feeding comprises the two connecting pieces 2 and 5. The first connecting piece 2 can be connected to the connection nozzle 3 of a feeding container 4. This connection is realized in the production process irreversibly. The second connecting piece 5 can be connected to the transfer unit 6, wherein said connecting piece 5 of the attachment is formed as a male “ENLock” connector, and the connecting piece 10 of the transfer unit is formed as a female “ENLock” connector. The second connecting piece 5 of the attachment further comprises a seal in the form of a flip-off cap 11.

As can be seen, the connection nozzle 3 is welded into the film weld seam of the food container 4. Therefore the connecting nozzle comprises a sealing membrane on its side away from the feeding container 16.

The attachment further comprises a means for opening the feeding container 7, which is designed as a short spike, and a cavity or channel 8 that connects the two connecting pieces 2 and 5 so that a food flow is made possible from the feeding container through the connection nozzle and the attachment to the transfer unit. The attachment 1 and the means for opening are made of plastic in one piece.

The connection nozzle 3 further comprises an external thread 9, which engages with the inner thread 12 of the attachment. Thus, in this embodiment, too, a rotation of the attachment 1 moves the means for opening the feeding container 7 axially in the direction of the first connecting piece 2 and, after penetrating the feeding container wall, it can be locked. Alternatively, also a bayonet mechanism can be used here to achieve the axial movement and its locking

The connection nozzle 3 also comprises a projection 13, which corresponds to a snap-in edge of the attachment 1 and makes possible an irreversible connection between the connection nozzle and the attachment during the production process. Furthermore, thus, the connection between the connection nozzle and the attachment is sealed and the contents of the feeding container are effectively protected against contamination.

FIG. 3B shows the same embodiment as FIG. 3A, after the nozzle attachment 1 was connected to the connecting nozzle 3. The connection was achieved by simply plugging the attachment on the connection nozzle in the production process, wherein the snap-in edge 14 of the attachment was pressed behind the projection 13 of the connection nozzle in order to ensure an irreversible bond.

It can also be seen that the internal thread 12 of the attachment does not yet completely engage into the external thread 9 of the connection nozzle and the means for opening the feeding container 7 is in a first defined position at the sealing means 16 without piercing it. In addition, the flip-off cap 11 is still attached to the second connecting piece 5.

In a next step, the flip-off cap can be now removed and the male “ENLock” connector 5 of the attachment can be safely and comfortably connected to the female “ENLock” connector 10 of the transfer unit 6 so as to provide a hermetically sealed food flow from the feeding container 4 through the means for opening 7 of the attachment 1, and further through the “EN-Lock” connection 5, 10 into the transfer system.

FIG. 3C shows the same embodiment as FIGS. 3A and 3B, after by a rotational movement of the attachment and by the interlocking of the two threads 9 and 12, the means for opening the feeding container 7 was moved axially in the direction of the feeding container 4 into a second defined position and the sealing means 16 was pierced.

In spite of the piercing of the sealing means 16, i.e., the opening of the feeding container, protection of the food in the container is protected from contamination, because before the penetration of the sealing means, the flip-off cap 11 was removed from the second connecting piece 5 and the transfer unit was connected by means of the “ENLock” connector.

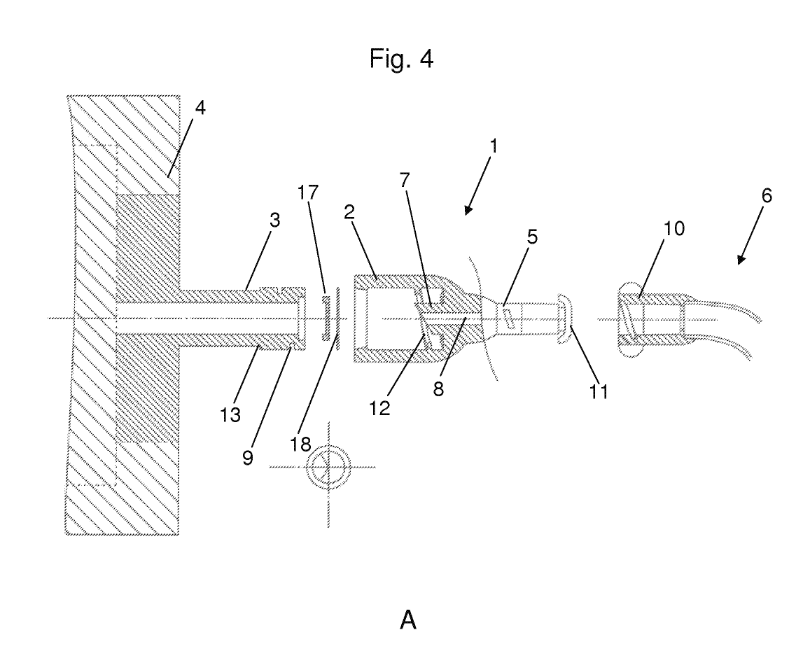

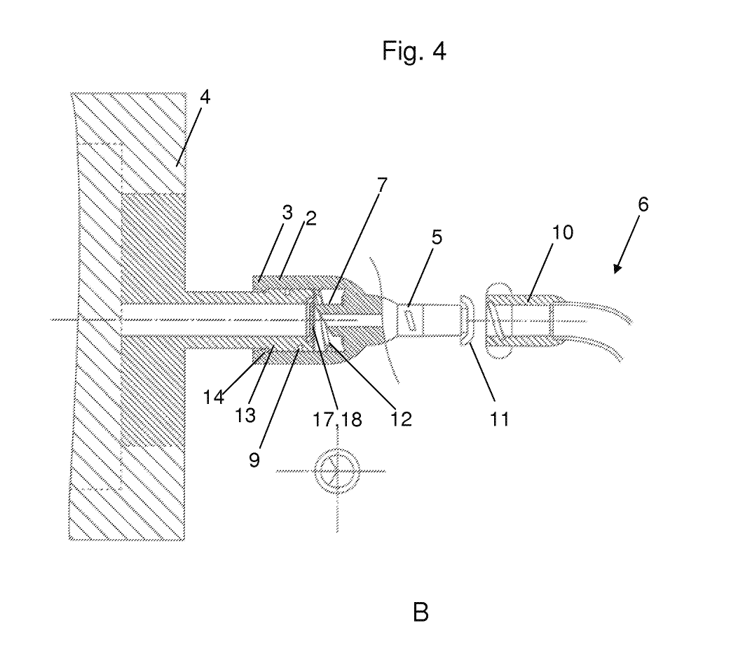

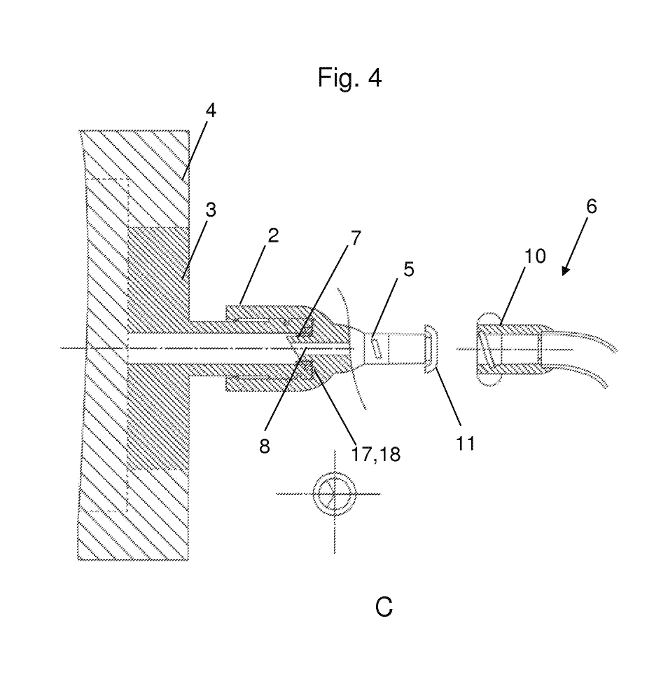

FIG. 4 shows a further embodiment of the invention attachment or device. The embodiment shown in FIG. 4 differs from the embodiment shown in FIG. 3 in that a membrane with a reversible valve function 17 and a sealing means 18 are mounted on the connection nozzle 3. The remaining reference symbols are the same.

The diaphragm valve 17 is inserted into the open end of the connection nozzle 5 and it has a reversible valve function. The sealing means 18 is an aluminum foil additionally applied over the membrane.

While FIG. 4A reproduces the state of the device prior to connecting the attachment to the connecting nozzle 3, FIG. 4B shows the same embodiment after the attachment 1 has been irreversibly connected to the connection nozzle 3 in the production process. The connection was accomplished by simply plugging the attachment on the connection nozzle, wherein the snap-in edge 14 of the attachment was pressed behind the projection 13 of the connection nozzle in order to ensure an irreversible bond.

It can also be seen that the internal thread 12 of the attachment does not yet completely engage into the external thread 9 of the connection nozzle and the means for opening the feeding container 7 is in a first defined position on the outer side of the sealing means 18 without piercing it. In addition, the flip-off cap 11 is still attached to the second connecting piece 5.

FIG. 4C shows the same embodiment as FIGS. 3A and 3B, after by a rotational movement of the attachment 1 and by the engagement of the two threads 9 and 12, the means for opening the feeding container 7 has been axially moved in the direction of the feeding container 4 into a second defined position and the sealing means 18 and the valve membrane 17 have been pierced.

Despite the piercing of the sealing means 18 and the valve membrane 17, i.e., despite the opening of the feeding container, protection from contamination of the food located in the food container is ensured, because the cap 11 has not been torn away from the second connecting piece 5 yet. Alternatively, the connection of the transfer unit to the “ENLock” connector is conceivable before piercing the sealing means 17. This has the advantage of creating a system that is already closed to the outside environment before the food flow starts.

In a next step, the flip-off cap can now be removed and the male “ENLock” connector 5 of the attachment can be safely and comfortably connected to the female “ENLock” connector 10 of the transfer unit 6 so as to provide a hermetically sealed food flow from the feeding container 4 through the opening means 7 of the attachment 1, and further through the “ENLock” connection 5, 10 into the transfer system.

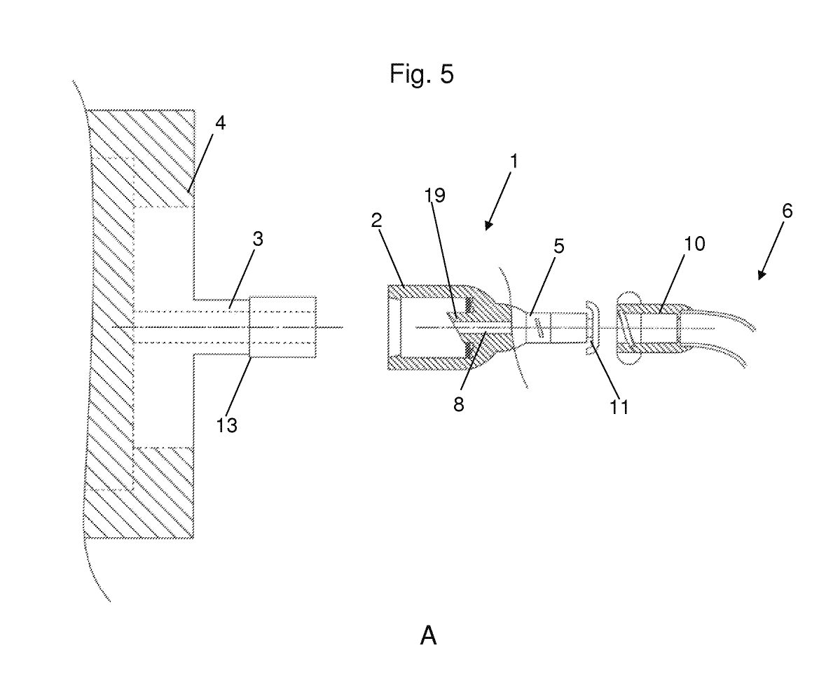

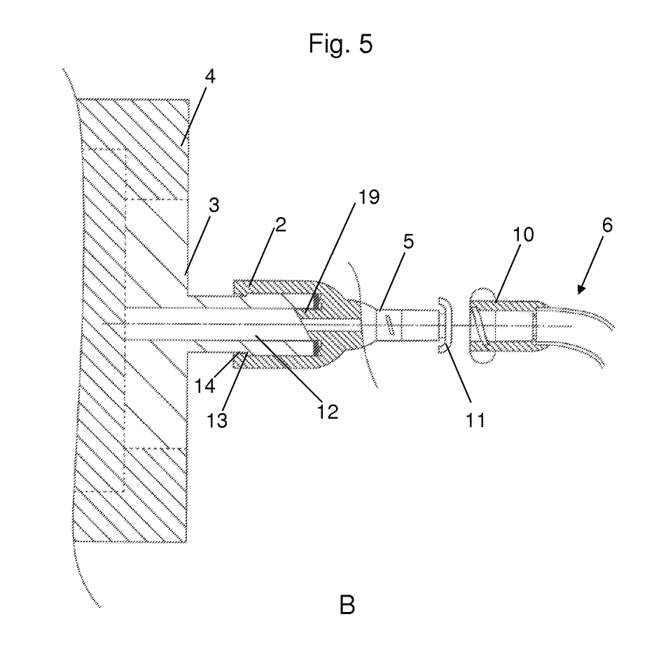

FIG. 5 shows yet another embodiment of the invented attachment or device.

As shown in FIG. 5A, the attachment 1 for a device for enteral feeding comprises the two connecting pieces 2 and 5. The first connecting piece 2 can be connected to the connection nozzle 3 of a feeding container 4. The second connecting piece 5 can be connected to the transfer unit 6, wherein this connecting piece 5 of the attachment is formed as an “EN-Lock” connector, and the connecting piece 10 of the transfer unit is configured as a female “ENLock” connector. The second connecting piece 5 of the attachment further comprises a sealing in the form of a flip-off cap 11.

As can be seen, the connection nozzle 3 is welded to the wall of the feeding container 4 and comprises a direct access to the contents of the food container. This can be the case, for example, because the connection nozzle had previously been closed by a sealing, such as a flip-off cap, which has already been removed.

Accordingly, the attachment does not need to comprise any means for opening the feeding container 7, as was the case in the embodiments described above. Nevertheless, a short spike 19 is formed integrated in the attachment 1, which comprises a cavity or channel 8 that connects the two connecting pieces 2 and 5 so that a food flow from the feeding container through the connection nozzle and the attachment to the transfer unit is made possible. The attachment 1 and spike are made of plastic in one piece.

Furthermore, the connection nozzle need not necessarily comprise an external thread, because in the attachment there is no movable means for opening the food container. However, such an external thread can be provided, e.g., to allow a screw as a sealing means for the connection nozzle.

The connection nozzle 3 comprises a projection 13, which corresponds to a snap-in edge of the attachment 1 and allows to create an irreversible connection between the connection nozzle and the attachment. Furthermore, the connection between the connection nozzle and the attachment is thus sealed and the contents of the feeding container are effectively protected from contamination.

FIG. 5B shows the same embodiment as FIG. 5A, after the attachment 1 has been connected to the connection nozzle 3. The connection he was renewed by simply plugging the attachment on the connection nozzle, wherein the snap-in edge 14 of the attachment was pressed behind the projection 13 of the connection nozzle in order to ensure an irreversible bond.

Furthermore, the flip-off cap 11 is still fastened on the second connecting piece 5 so that in spite of the open feeding container, protection of the food located in the food container from contamination is ensured.

In a next step, the flip-off cap can now be removed and the male “ENLock” connector 5 of the attachment can be safely and comfortably connected to the female “ENLock” connector 10 of the transfer unit 6 so as to provide a hermetically sealed food flow from the feeding container 4 through the spike 19 of the attachment 1, and further through the “ENLock” connection 5, 10 to the transfer system.

1. An attachment for a device for enteral feeding, comprising at least two connecting pieces, wherein the first connecting piece can be connected to the connection nozzle of a feeding container and the second connecting piece can be connected to a transfer unit so that a flow of food from the feeding container and the connection nozzle and the attachment to the transfer unit is possible, the attachment including a mechanism for opening the feeding container.

2. The attachment according to claim 1, wherein the first connecting piece allows an irreversible connection between the attachment and the connection nozzle.

3. The attachment according to one claim 1 wherein the second connecting piece comprises a locking element.

4. The attachment according to claim 1 wherein the second connecting piece is formed as an ENLock connector.

5. The attachment according to claim 1, wherein the mechanism for opening the feeding container is formed as a spike.

6. The attachment according to claim 1, wherein the mechanism for opening can be moved in the direction of the first connecting piece.

7. The attachment according to claim 1, wherein the attachment comprises means for sealing at least one of the connecting pieces.

8. A device for enteral feeding comprising an attachment according to the preceding claims and a feeding container having attached thereto a connection nozzle, and optionally, a transfer unit, wherein the attachment and the connection nozzle are connected by the first connecting piece, and wherein the attachment and the transfer unit are connected by the second connecting piece of the attachment.

9. The device according to claim 8, wherein the attachment and the connection nozzle are irreversibly connected to each other and/or wherein the transfer unit and the attachment are positively connected to each other by the second connecting piece of the attachment.

10. The attachment for a device for enteral feeding, comprising at least two connecting pieces, wherein the first connecting piece can be connected to the connection nozzle of a feeding container, and the second connecting piece can be connected to a transfer unit so that a flow of food from the feeding container through the connection nozzle and the attachment into the transfer unit is possible, wherein the second connecting piece is formed as an ENLock connector.
